(19) United States
(12) Plant Patent Application Publication
(10) Pub. No.: US 2011/0302681 P1
(43) Pub. Date: Dec. 8, 2011

(54) Dianella tasmanica plant named 'Lemon Lime'

(50) ;

(76) Inventor: Joseph Roberts, Miami, FL (US)

(21) Appl. No.: 12/802,328

(22) Filed: Jun. 4, 2010

(51) Int. Cl. A01H 5/00

(52) U.S. Cl. PLT/424

(57) ABSTRACT

‘Lemon Lime’ is a distinctive variety of Dianella caerulea which is characterized by the combination of its short to medium plant height, dense upright growth habit with flowers positioned just above the foliage, upright leaf attitude with distal weeping, year round flowering season, and violet blue flower color with violet blue berry color.

### LATIN NAME OF THE GENUS AND SPECIES

The Latin name of the novel variety disclosed herein is Dianella tasmanica.

### VARIETY DENOMINATION

The inventive variety of Dianella tasmanica disclosed herein has been given the varietal denomination ‘Lemon Lime’.

### BACKGROUND OF THE INVENTION

The present invention relates to a new and distinct sport from Dianella tasmanica “Variegata”, which has been given the varietal denomination of ‘Lemon Lime’. It is distinguished by lemon lime variegation on green leaves. Its market class is that of ornamental plants, both as a potted plant and as an in-ground landscape plant. ‘Lemon Lime’ is intended for use in landscaping and as a decorative potted plant.

Parentage.

The Dianella tasmanica ‘Lemon Lime’ was first discovered in early 2008 in a farm in Germania, Costa Rica. Dianella tasmanica ‘Lemon Lime’ was selected as a naturally occurring sport from an established field of from Dianella tasmanica “Variegata”.

Asexual reproduction.

‘Lemon Lime’ was first asexually propagated by division in 2008, in Germania, Costa Rica. It was asexually reproduced 2008 through the present, and confirmed to be stable in character. The distinctive characteristics of the inventive variety, ‘Lemon Lime’, have remained stable and true to type from generation to generation through successive cycles of asexual reproduction via vegetative division.

### SUMMARY OF THE INVENTION

‘Lemon Lime’ is a distinctive variety of Dianella tasmanica which is characterized by its lemon lime variegation on green leaves, thus producing a uniquely distinct coloration.

### BRIEF DESCRIPTION OF THE FIGURES

The photographs in the drawings were made using conventional techniques and show the colors as true as reasonably possible by conventional photography. Colors in the photographs may differ slightly from the colour values cited in the detailed botanical description, which accurately describe the colors of the new Dianella tasmanica ‘Lemon Lime’.

FIG. 1 shows several ‘Lemon Lime’ plants in flower.

FIG. 2 shows several ‘Lemon Lime’ plants. the stem and leaf basil sheath of ‘Lemon Lime’.

FIG. 3 shows a ‘Lemon Lime’ panicle.

FIG. 4 shows an extreme branch of a ‘Lemon Lime’ panicle showing several flower buds.

FIG. 5 shows a close-up view of a number of ‘Lemon Lime’ flower buds.

FIG. 6 shows several ‘Lemon Lime’ flower buds, and two ‘Lemon Lime’ flowers in full bloom.

### DETAILED BOTANICAL DESCRIPTION OF THE PLANT

The following is a detailed botanical description of a new and distinct variety of a Dianella tasmanica ornamental plant known as ‘Lemon Lime’. The descriptions disclosed herein are based upon observations of the plant grown in soil and pots in Germania, Costa Rica, since early 2008.

Those skilled in the art will appreciate that certain characteristics will vary with older or, conversely, younger plants. ‘Lemon Lime’ has not been observed under all possible environmental conditions. Where dimensions, sizes, colors and other characteristics are given, it is to be understood that such characteristics are approximations or averages set forth as accurately as practicable. The phenotype of the variety may differ with variations in the environment such as season, temperature, light intensity, day length, cultural conditions and the like.

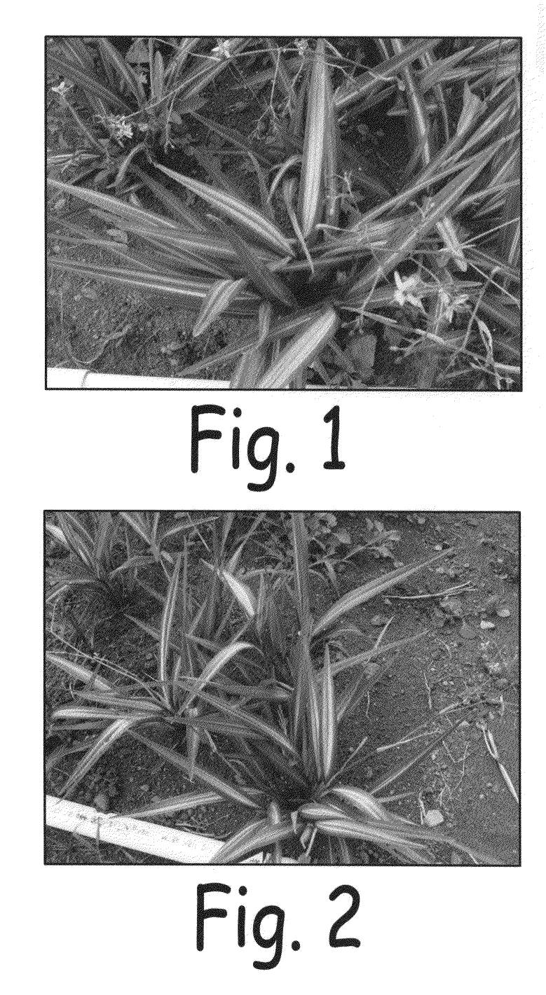

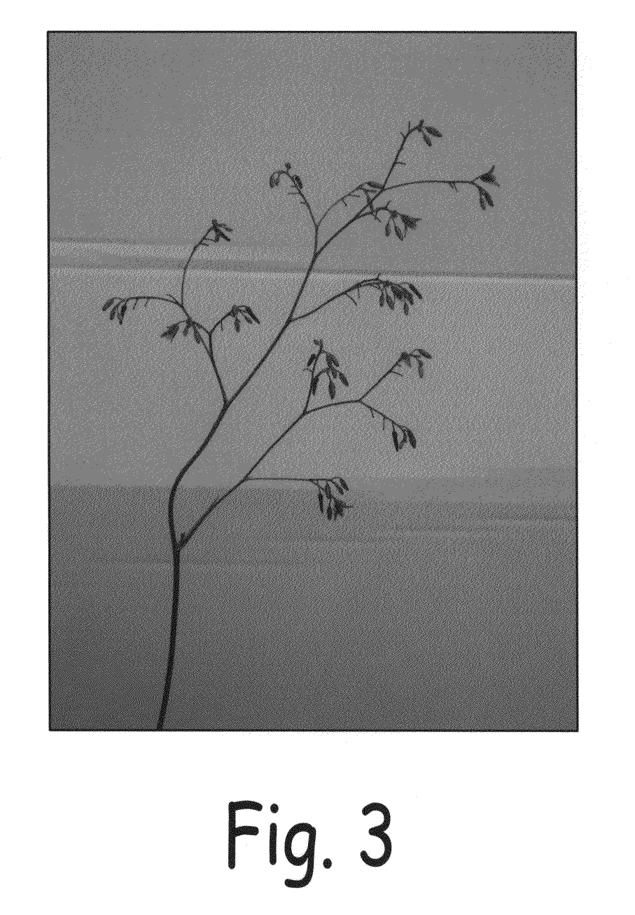

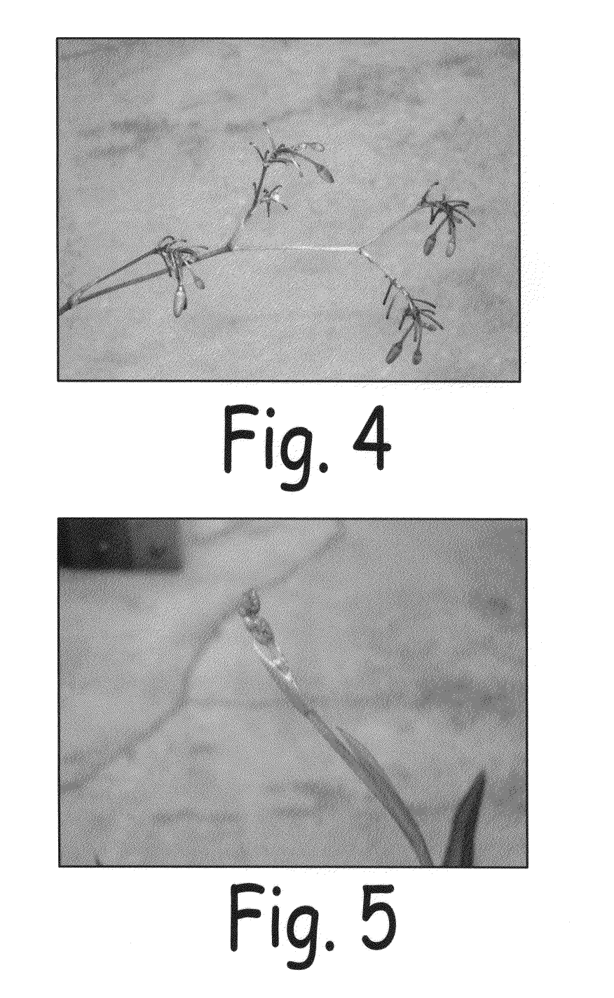

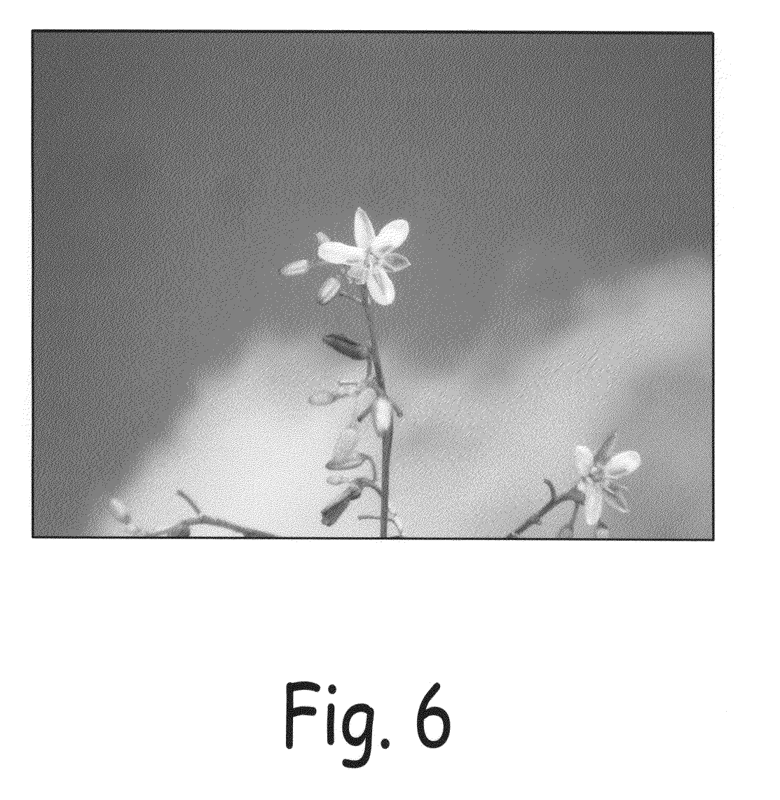

‘Lemon Lime’ is a Dianella tasmanica sport, which was discovered at an established Dianella tasmanica field cultivated in Germania, Costa Rica. After its selection, ‘Lemon Lime’ was asexually propagated by vegetative division. ‘Lemon Lime’ is distinguished by its distinct leaf variegation, which differentiates it from other Dianella tasmanica plants. A botanical description of ‘Lemon Lime’ is provided below. Representative Dianella tasmanica ‘Lemon Lime’ plants are shown in FIGS. 1 and 2.
Technical description of the variety.
   Growth habit.—The mature ‘Lemon Lime’ plant is 40 to 50 cm high, with a spread of 40-50 cm. Its leaves emerge at ground level from a central point, and extend radially outwards, and upwards. Shoots grow laterally from the base of the main shoot. The cuttings are rhizome offsets, and take ten to fourteen days to initially produce roots. The young ‘Lemon Lime’ plants then will become rooted plants in eight weeks at 22 degrees Centigrade. The roots are thick yellow leaders with thin yellow side branches. The root habit is vertical leaders with lateral side branches.
   Roots.—A central root system sends out radial stoliniferous rhizomes, which produce new plants. The roots themselves are thick yellow vertical leaders, with thinner yellow lateral side-branches.
   Stem.—‘Lemon Lime’ leaves emerge at ground level from a central point, and extend radially outwards, and upwards. Shoots grow laterally from the base of the main shoot.
   Foliage.—The lanceolate ‘Lemon Lime’ leaves emerge alternately from the base of the main shoot. They measure approximately 35 cm in length and 3 cm in width. The leaf apex is narrowly linear; the base attenuate. The leaf margin is entire. Both top and bottom leaf texture is smooth, while the leaf edges are slightly course. Leaf veination is parallel. Upper leaf surface: The upper surface of the leaf is green, with a broad, longitudinal center stripe. The color of the broad, longitudinal center stripe is white at the proximal end, transitions through cream at the leaf midpoint, and then to light green at the leaf distal end. A few parallel longitudinal narrow Nile green lines run longitudinally along the center stripe. Lower leaf surface: The lower surface of the leaf is green, with a broad, longitudinal center stripe. The color of the broad, longitudinal center stripe is lime green at the proximal end, transitions through dull lime green at the leaf midpoint, and then to light green at the leaf distal end. A few parallel longitudinal narrow Nile green lines run longitudinally along the center stripe.
   Panicle.—As may be observed in FIGS. 1 and 2, and in more detail in FIGS. 3-5, panicles grow from the ‘Lemon Lime’ central stalk, measuring up to 72 cm in height and 16 cm wide. Flowers grow on the panicle, which may have 16 branches of four to five flowers and buds each. A freely flowering plant may have sixty-plus flowers and buds.
   Flowers.—As is visible in FIG. 6, each plant grows numerous single flowers, which grow from the shoot terminal. Petals are free, with ovary superior. It is believed that the flowering season is during Spring and Summer. ‘Lemon Lime’ flowers are approximately 1.3 cm in diameter, with a depth of around 1 cm. Flower buds are around 0.9 cm long, with a diameter of 0.3 cm, and are olive green (Exotica). Petals are palmate, and generally there are three white/light blue petals per flower, with length of approximately 0.8 cm and width of 0.4 cm. Flower apex is obtuse, and base obtuse. The margin is entire. Each flower has three sepals, olives/gray in color. The female pistil is white, and the male stamen is yellow.
   Seeds and/or fruit.—None observed.
‘Lemon Lime’ compared to other varieties of dianella caerulea and dianella brevipedunculata The ‘Lemon Lime’ leaf coloration is its most distinguishing characteristic relative to other Dianella tasmanica sports. In particular, the upper surface of the leaf is green, with a broad, longitudinal center stripe. The color of the broad, longitudinal center stripe is white at the proximal end, transitions through cream at the leaf midpoint, and then to light green at the leaf distal end. A few parallel longitudinal narrow Nile green lines run longitudinally along the center stripe. The lower surface of the leaf is green, with a broad, longitudinal center stripe. The color of the broad, longitudinal center stripe is lime green at the proximal end, transitions through dull lime green at the leaf midpoint, and then to light green at the leaf distal end. A few parallel longitudinal narrow Nile green lines run longitudinally along the center stripe.

That which is claimed is:

1. A new and distinct variety of Dianella tasmanica plant named ‘Lemon Lime’, substantially as described and illustrated herein.
